(19) United States
(12) Patent Application Publication
(10) Pub. No.: US 2023/0310671 A1
(43) Pub. Date: Oct. 5, 2023

(54) Elongated Germicidal Lighting Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/711,306

(22) Filed: Apr. 1, 2022

(51) Int. Cl. A61L 2/10; H05B 45/28

(52) U.S. Cl. CPC A61L 2/10; H05B 45/28; A61L 2202/14; A61L 2202/11

(57) ABSTRACT

An elongated germicidal lighting device includes an elongated housing, a first light source, a second light source, a third light source, a first driver, a second driver, a third driver, and a controller. The first light source shines a visible light above the housing whereas the second light source shines visible light below the housing. The third light source is an ultraviolet-C (UVC) light source emitting germicidal irradiation to disinfection ambient air and a surface in line of sight. The controller is configured to turn on the first light source, the second light source, and the third light source individually or any combination thereof.

### BACKGROUND

### Technical Field

The present disclosure pertains to germicidal lighting devices and, more specially, proposes an elongated germicidal lighting device.

### Description of Related Art

During the COVID-19 pandemic, there is a growing need for adding new disinfecting equipment to facilities for disinfecting air, or surfaces, or both to provide a healthier indoor environment. Stationary disinfecting equipment is preferred over mobile disinfecting equipment for the latter tends to be more labor intensive. Stationary disinfecting equipment may be a dedicated disinfecting equipment or a hybrid equipment. The present disclosure presents an elongated germicidal lighting device that is a hybrid equipment for it provides general lighting and germicidal irradiation for disinfecting ambient air and surfaces in line of sight.

### SUMMARY

In one aspect, the germicidal lighting apparatus comprises an elongated housing, a first elongated light source emitting a visible light, a second elongated light source emitting a visible light, a third light source emitting an ultraviolet-C (UVC) wavelength in the range of 200˜280 nm, a first driver, a second driver, a third driver, and a controller. The housing houses the first light source, the second light source, the third light source, the first driver, the second driver, the third driver, and the controller. The first driver is configured to convert an external power to a first internal power to activate the first light source. The second driver is configured to convert the external power to a second internal power to activate the second light source. The third driver is configured to convert the external power to a third internal power to activate the third UVC light source. Moreover, the first light source is configured to shine a light above the housing, whereas the second light source is configured to shine a light below the housing. The third light source is configured to emit germicidal irradiation and disinfect ambient air and a surface in line of sight. The controller is configured to turn on the first light source, the second light source, and the third light source individually, or any combination thereof.

The distinction between the first driver and the second driver is logical, not physical. While they may be implemented via two physical drivers, it is possible to use one physical driver to power the first light source and the second light source. In some embodiments, the first driver and the second driver are embodied in one physical driver circuitry. This physical driver circuitry may have two output power ports one for powering the first light source and the other for powering the second light source. Alternatively, this physical driver circuitry may have one output power port that drives both the first and the second light sources in parallel, and the controller is configured to determine whether the first light source or the second light source or both would be turned on. Similarly, in some embodiments, the first driver, the second driver, and the third driver are embodied in one physical driver circuitry, and the controller is configured to determine whether the first light source or the second light source or the third light source or any combination of them will be turned on.

In some embodiments, the housing is configured to surface-mounted on a wall. Therefore, it may be used on a wall or a flat surface above a patient bed in a patient room at a hospital. Under this scenario, the first light source is used effectively as an up light for providing ambient light, whereas the second light source is used as a down light for reading. The third light source provides germicidal irradiation to ambient air and surfaces in line of sight in the patient room.

The third UVC light source is not required to be a particular shape. In some embodiments, the third light source is of an elongated shape. The third light source with an elongated shape provides a larger area coverage as compared to a non-elongated UVC light source.

In some embodiments, the controller is configured to support two operation modes comprising a germicidal mode and a non-germicidal mode. In the germicidal mode, the controller is configured to turn on the third light source, whereas in the non-germicidal mode, the controller is configured to turn off the third light source.

In addition to the support of the germicidal mode operation, it would be useful to support different operation durations for the germicidal mode. For example, a shorter duration for 5 minutes may be used for a quick disinfection, and a longer duration for 10 minutes may be used for a thorough disinfection. Therefore, in some embodiments, the controller is further configured to support a first operation duration and a second operation duration for the germicidal mode operation, wherein the second operation duration is longer than the first operation duration.

During the germicidal mode operation, the third light source emits UVC irradiation into the environment. If an occupant enters the environment during the germicidal mode operation of the device, the occupant may be subject to UVC exposure, posing a health risk. It is therefore necessary to safeguard the germicidal mode operation properly and reduce the chances of UVC exposure to an occupant. In some embodiments, the device further includes a motion sensor. During the germicidal mode operation, when a motion is detected by the motion sensor, the controller is configured to suspend the germicidal mode operation. When no motion is detected by the motion sensor, the controller is configured to resume the germicidal mode operation.

In some embodiments, the controller is configured to support two lighting modes comprising a regular lighting mode and a boost lighting mode. In the regular lighting mode, the controller is configured to turn on either the first light source or the second light source or both. In the boost lighting mode, the controller is configured to turn on either the first light source or the second light source or both at a light output that is 50% or more than the light output in the regular lighting mode. During the boost lighting mode, the controller may boost the light output of both the first and the second light sources. Alternatively, the controller may boost only the light output of the second light source. The regular lighting mode is used for general conditions. The boost lighting mode is used when a healthcare personnel needs to examine a patient in a patient room where a device of the present disclosure in installed. The boost lighting mode operation provides a brighter light for the healthcare personnel to better examine the patient.

In some embodiments, the first driver is a dimmable driver configured to dim the first light source, and the second driver is a dimmable driver configured to dim the second light source.

In some embodiments, the first light source and the second light source each supports more than one color temperatures, and the controller is configured to tune the color temperature of the first light source and the color temperature of the second light source.

In some embodiments, the first light source and the second light source each comprises multiple light emitting diodes (LEDs).

The third light source may be a UVC tubular lamp. In some embodiments, the third light source comprises multiple UVC LEDs. In some other embodiments, the third light source comprises one or more excimer lamps.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1(a) schematically depicts the exterior of an embodiment of the present disclosure with a front view and two side views.

FIG. 1(b) schematically depicts the same embodiment from an angle.

FIG. 1(c) schematically depicts the same embodiment without the diffuser lens for the up light and the down light.

FIG. 1(d) schematically depicts the same embodiment with a look-through view showing the three drivers and the controller.

FIG. 1(e) schematically depicts a cross section view of the embodiment.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of elongated germicidal lighting device.

The present disclosure includes an elongated housing, a first light source, a second light source, a third light source, a first driver, a second driver, a third driver, and a controller. The first light source shines a visible light above the housing whereas the second light source shines a visible light below the housing. The third light source is an ultraviolet-C (UVC) light source emitting germicidal irradiation to disinfection ambient air and a surface in line of sight. The controller is configured to turn on the first light source, the second light source, and the third light source individually or any combination thereof.

### Example Implementations

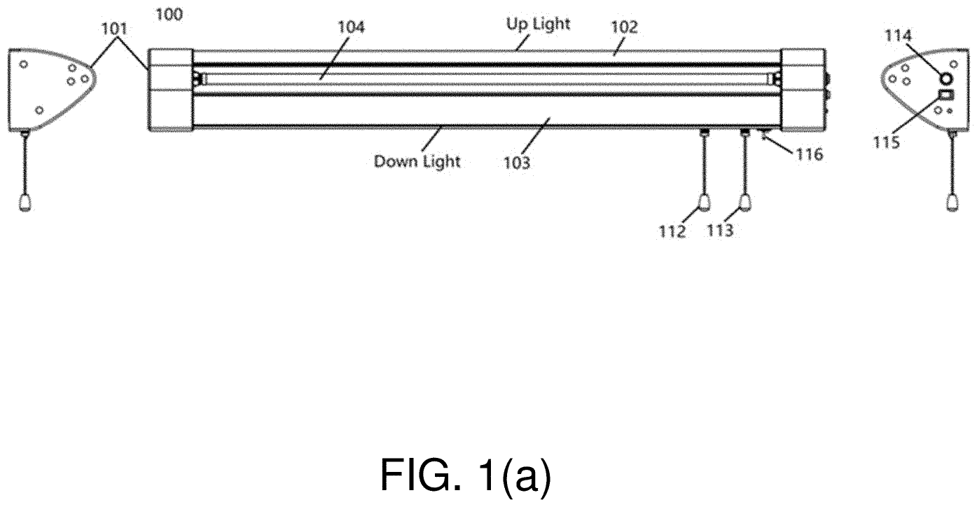

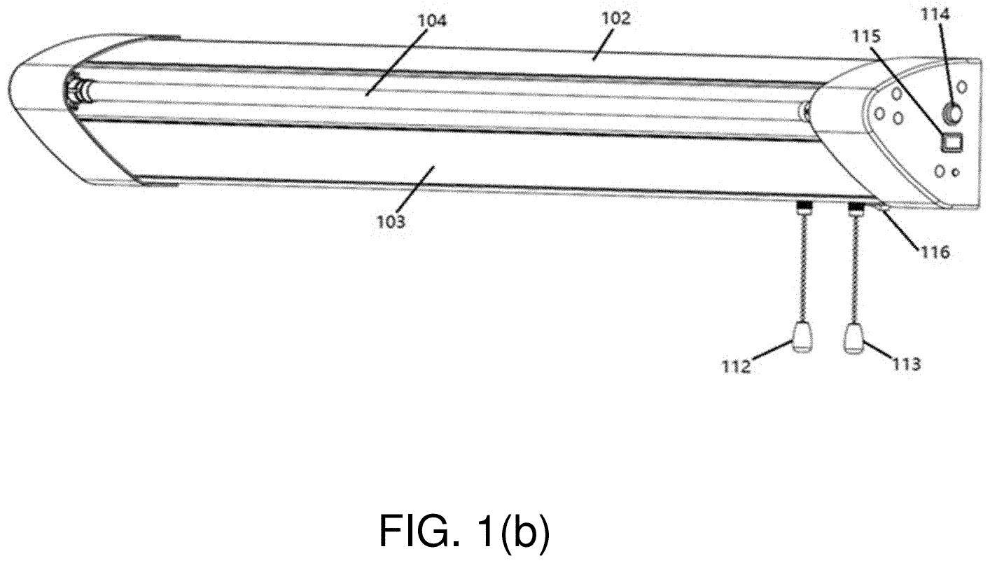

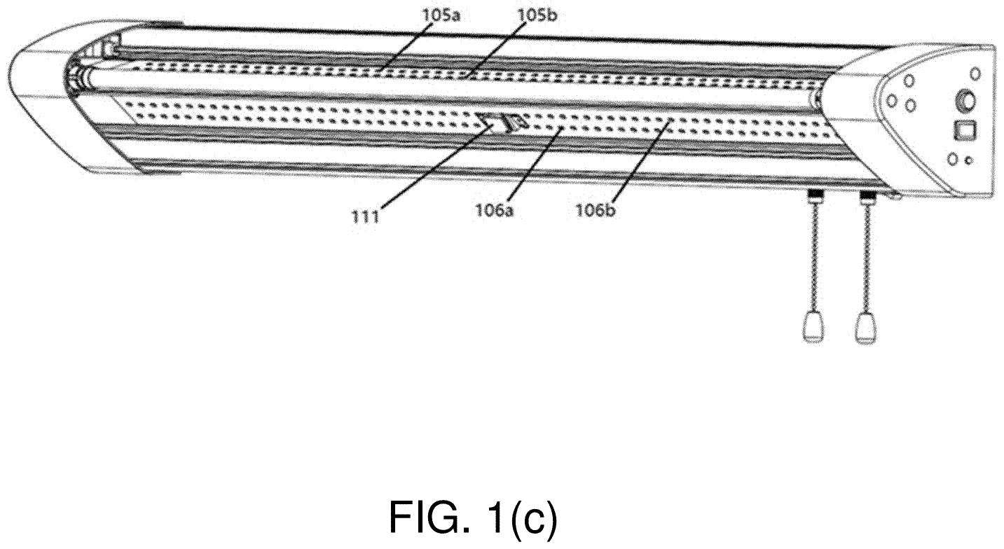

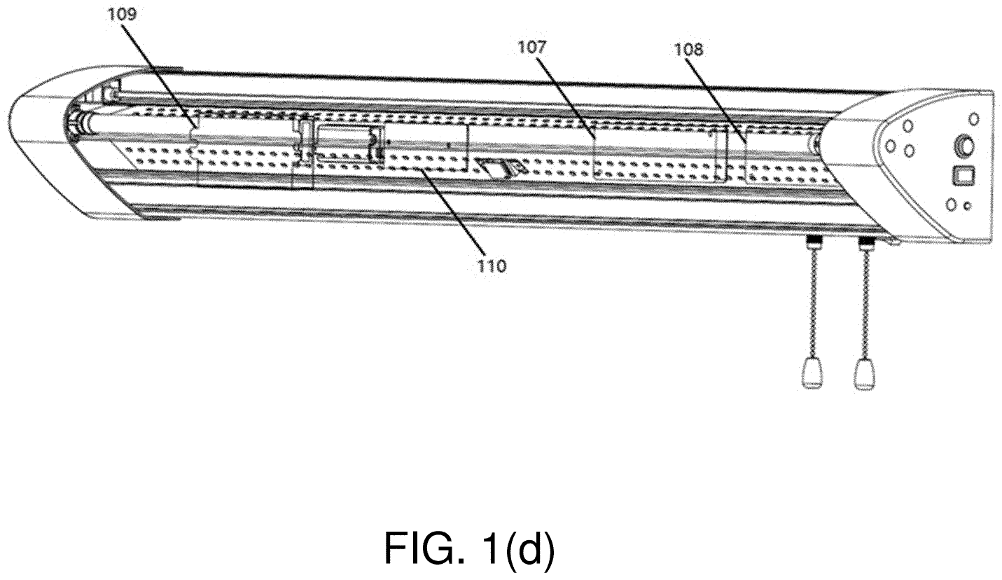

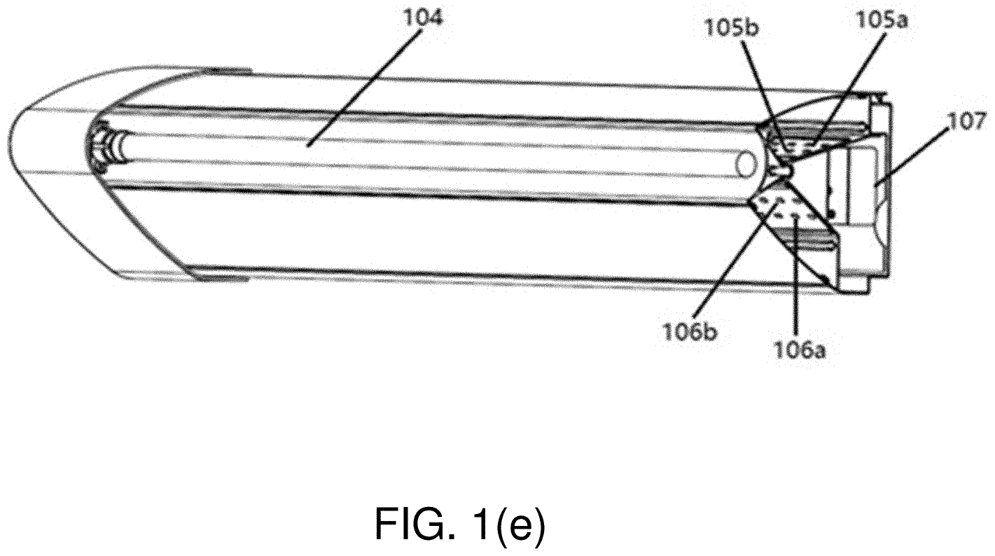

FIG. 1 schematically depicts an embodiment of the present disclosure 100. FIG. 1(a) schematically depicts the exterior of the embodiment with a front view and two side views. FIG. 1(b) depicts the same embodiment from an angle. FIG. 1(c) schematically depicts the same embodiment without the diffuser lens for the up light and the down light. FIG. 1(d) schematically depicts the same embodiment with a look-through view showing the three drivers 107, 108, 109 and the controller 110. FIG. 1(e) schematically depicts a cross section view of the embodiment.

The housing 101 houses all other components of the device. It may be surface-mounted on a wall or a flat surface. The diffuser lens 102 is for the up light and the diffuser lens 103 is for the down light. FIG. 1(c) schematically depicts the same embodiment with the lenses 102 and 103 removed. The up light shines its light above the housing 101 and includes the first light source comprising two rows of LED, 3000K LEDs 105a an 6000K LEDs 105b. The down light shines its light below the housing 101 and includes the second light source comprising two rows of LED, 3000K LEDs 106a an 6000K LEDs 106b. The pull chain 112 is used to notify the controller 110 to turn on the first light source and to change its color temperature to 3000K, or 6000K, or 4500K (by mixing 3000K and 6000K LEDs). Similarly, the pull chain 113 is used to notify the controller 110 to turn on the second light source and to change its color temperature.

A UVC T5 tubular lamp 104 is used as the third light source. A push-style on/off switch 114 is used for activating the germicidal mode operation. When the switch 114 is pushed, the controller 110 turns on the UVC T5 lamp 104 and operates in the germicidal mode based on the duration setting of the switch 115. The switch 115 is used to set the operation duration for the germicidal mode between 5 minutes for a quick disinfection or 10 minutes for a thorough disinfection. Upon the completion of the germicidal operation, the switch 114 is reset to the off state automatically. If during the germicidal mode operation, a motion is detected by the motion sensor 111, the controller suspends the germicidal mode operation. Once no further motions are detected by the motion sensor 111, the controller 110 resumes the germicidal mode operation.

When the Exam mode switch 116 is activated, the controller 110 operates the first light source 105a, 105b and the second light source 106a,106b in the boost lighting mode at 3000 lumen light output each. When the Exam mode switch 116 is not activated, the controller 110 operates the first light source 105a and 105b and the second light source 106a and 106b in the regular lighting mode where the first light source or the second light source only produces 2000 lumen.

The first driver 107 converts an external power to an internal power to drive the first light source 105a and 105b. The second driver 108 converts an external power to an internal power to drive the second light source 106a and 106b. The ballast 109 acts as the third driver for converting an external power to an internal power to power the third UVC light source 104. Though the dimming function is not shown, it may be achieved by using dimmable drivers for drivers 107, 108 with additional dimming control lines connecting to external dimmer(s).

The UVC T5 lamp 104 is used as example for the third light source. It may be replaced by UVC LEDs (with a suitable driver as the third driver) or a far UVC excimer lamp (with a suitable ballast as the third driver).

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. An elongated germicidal lighting device, comprising:
an elongated housing;
a first elongated light source configured to emit a first visible light;
a second elongated light source configured to emit a second visible light;
a third light source configured to emit an ultraviolet-C (UVC) wavelength in a range of 200˜280 nm;
a first driver;
a second driver;
a third driver; and
a controller,
wherein:
the housing is configured to house the first light source, the second light source, the third light source, the first driver, the second driver, the third driver, and the controller,
the first driver is configured to convert an external power to a first internal power to activate the first light source,
the second driver is configured to convert the external power to a second internal power to activate the second light source,
the third driver is configured to convert the external power to a third internal power to activate the third light source,
the first light source is configured to shine the first visible light above the housing,
the second light source is configured to shine the second visible light below the housing,
the third light source is configured to emit a germicidal irradiation to disinfect an ambient air and a surface in a line of sight, and
the controller is configured to turn on the first light source, the second light source, and the third light source individually or in any combination thereof.

2. The device of claim 1, wherein the first driver and the second driver are embodied in one physical driver circuitry.

3. The device of claim 1, wherein the first driver, the second driver, and the third driver are embodied in one physical driver circuitry.

4. The device of claim 1, wherein the housing is configured to be surface-mounted on a wall or a flat surface.

5. The device of claim 1, wherein the third light source has an elongated shape.

6. The device of claim 1, wherein the controller is configured to support two operation modes comprising a germicidal mode and a non-germicidal mode such that:
in the germicidal mode, the controller is configured to turn on the third light source, and
in the non-germicidal mode, the controller is configured to turn off the third light source.

7. The device of claim 6, wherein the controller is further configured to support a first operation duration and a second operation duration for the germicidal mode operation, and wherein the second operation duration is longer than the first operation duration.

8. The device of claim 6, further comprising:
a motion sensor coupled to the controller and configured to operate in conjunction with the controller,
wherein, during the germicidal mode:
when a motion is detected by the motion sensor, the controller is configured to suspend the germicidal mode operation, and
when no motion is detected by the motion sensor, the controller is configured to resume the germicidal mode operation.

9. The device of claim 1, wherein the controller is configured to support two lighting modes comprising a regular lighting mode and a boost lighting mode such that:
in the regular lighting mode, the controller is configured to turn on either or both of the first light source and the second light source, and
in the boost lighting mode, the controller is configured to turn on either or both of the first light source and the second light source at an increased light output that is 50% or more than a light output in the regular mode.

10. The device of claim 1, wherein the first driver comprises a dimmable driver configured to dim the first light source, and wherein the second driver comprises a dimmable driver configured to dim the second light source.

11. The device of claim 1, wherein the first light source and the second light source each supports more than one color temperatures, and wherein the controller is configured to tune a first color temperature of the first light source and a second color temperature of the second light source.

12. The device of claim 1, wherein the first light source and the second light source each comprises multiple light emitting diodes (LEDs).

13. The device of claim 1, wherein the third light source comprises multiple UVC light emitting diodes (LEDs).

14. The device of claim 1, wherein the third light source comprises one or more excimer lamps.
